(19) United States
(12) Patent Application Publication
(10) Pub. No.: US 2025/0154097 A1
(43) Pub. Date: May 15, 2025

(54) METHOD FOR PRODUCING ISOCYANATES

(71) Applicant: Covestro Deutschland AG, Leverkusen (DE)

(72) Inventors: Mark Jacob, Monheim (DE); Guido Grommes, Troisdorf (DE); Thomas Schmitz, Leverkusen (DE); Pascal Scherpian, Moers (DE); Christian Jens, Köln (DE); Michael Merkel, Düsseldorf (DE); Stefanie Lang, Bochum (DE)

(21) Appl. No.: 18/834,261

(22) PCT Filed: Feb. 3, 2023
(86) PCT No.: PCT/EP2023/052653

§ 371 Date: Jul. 30, 2024

(30) Foreign Application Priority Data
Feb. 10, 2022 (EP) 22156145.9

(51) Int. Cl. C07C 263/10; C07C 263/20

(52) U.S. Cl. CPC C07C 263/10; C07C 263/20

(57) ABSTRACT

The invention relates to a method for producing isocyanates by reacting at least one amine or the salt thereof with a stoichiometric excess of phosgene in the condensed phase in the presence of a solvent, having the steps of: A) transitioning to a hot phosgenation process and B) expelling remaining phosgene and/or hydrogen chloride. The invention is characterized in that the reaction is carried out in batches in at least two reactors which are arranged parallel to one another and in that step A) and/or step B) is carried out asynchronously in at least two of the reactors arranged parallel to one another.

Isocyanates are important raw materials in the chemical industry. They are often produced in large amounts. Di-and polyisocyanates in particular are used primarily as starting materials for the production of polyurethanes. Their large-scale production is usually accomplished in good yields by phosgenation of the corresponding amines, amine hydrochlorides and/or amine carbonates with an excess of phosgene. In this reaction, the corresponding carbamoyl chloride is formed as an intermediate, which is then converted into the isocyanate with the elimination of hydrogen chloride.

The solvents for the reaction that have become established in industry are chlorobenzene or o-dichlorobenzene, which are largely inert in their behavior and highly suitable for the recovery of excess phosgene and separation thereof from hydrogen chloride. However, it is also possible to use other solvents that are inert under the reaction conditions.

The process offgas, which consists essentially of phosgene, hydrogen chloride and solvent, must be treated to avoid the release of phosgene. To increase the economic efficiency of the processes, it is also expedient to process the process offgas, which consists essentially of phosgene, hydrogen chloride and solvent, so that at least a portion of these constituents can be used further or reused. It is particularly advantageous when the phosgene can be used further or reused.

US2007/0249859A1 describes an at least 2-step sequence of absorption steps, the sequence comprising at least one isothermal absorption step and at least one adiabatic absorption step, with the phosgene thus obtained returned to the phosgenation reaction.

In DE102008009761A1, the process offgas stream initially undergoes partial condensation, with the liquid condensate processed in a stripping column. This process affords a liquid solvent stream in the bottoms and a gas stream at the head of the column that is directed into an absorption together with the hitherto uncondensed fractions. The phosgene present therein is absorbed in a solvent and the resulting phosgene solution reused in the phosgenation reaction, optionally after enrichment with further phosgene.

US2018/0044179A1 is based on a distillative separation of the process offgas. In this case, the process offgas stream is directed into a distillation column and a phosgene-containing stream withdrawn from the bottom thereof. At the head of the column, a stream consisting essentially of hydrogen chloride is withdrawn, compressed and so partially condensed. The condensed fraction is depressurized and fed back at the head of the column.

While phosgene is thus often recycled into the same process, there are other options for utilizing the hydrogen chloride. For example, it can be used or marketed simply as an aqueous solution, i.e. as hydrochloric acid. Alternatively, it can be catalytically or electrochemically oxidized to chlorine or used in the oxychlorination of ethylene to ethylene dichloride.

WO2008049783A1 describes a continuous process for producing isocyanates in which the mixing of the reactant streams and/or the reaction of the reaction mixture obtained takes place in at least two lines connected in parallel, thus making it possible, in the case of only part-load operation, for individual lines to be shut down and the lines in operation to continue running in the optimum range.

Many isocyanates are produced in large amounts and continuous processes are preferred for this. Isocyanates that are produced in smaller amounts are usually produced by batchwise liquid-phase phosgenation, since the costs involved in switching to a continuous process are very high and are not recoverable given the small amounts to be produced. In some cases, hybrid processes are also operated in which a batchwise reaction regime is combined with continuously operated downstream processing of the isocyanates produced. This requires the installation of buffer containers into which the reaction products are discharged and from which the continuous processing is then supplied.

For batchwise production of isocyanates too, treatment of the offgas and-where possible-recovery of phosgene from this offgas is also desirable. There are however problems with this that have yet to be addressed in the prior art. For example, the process offgas mass flows and composition thereof vary greatly depending on the operating conditions and, while it is relatively easy to install a buffer tank for liquid product streams, thus ensuring a uniform inflow stream for processing, this is not easily achievable for the process offgas on account of the large volumes and the toxic and corrosive constituents. During a batchwise phosgenation process, periods with very high mass flows of process offgas occur. In order to reliably avoid incomplete absorption of phosgene in, for example, a phosgene absorption according to DE102008009761A1 or US2007/0249859A1 and thus the penetration of phosgene into the HCl offgas from phosgene absorption, the absorption column and also the mass flow of solvents would have to be designed for these peaks with very high mass flows of process offgas. Outside these peak loadings the absorption column would then be oversized, which would in turn cause further problems.

U.S. Pat. No. 4,233,267A describes a system combining a batch reactor and a continuous distillation apparatus, wherein the volatile reaction products are first condensed and temporarily stored in a condensate container, from which they are fed into the continuously operated column essentially independently of the operating parameters of the batch reactor. For phosgenation processes, this approach has various disadvantages. Firstly, the condensation of phosgene, let alone HCl, requires very low temperatures. Secondly, the buffering that is necessary means that such an approach inevitably increases the amount of phosgene in the system.

It was thus an object of the invention to provide an improved process with which, even in batchwise production of isocyanates by phosgenation of amines, reprocessing of the process offgas can be operated efficiently and the recovered phosgene as far as possible reused in the phosgenation process.

This object was achieved by a process for producing isocyanates by reacting at least one amine or salt thereof with a stoichiometric excess of phosgene in the condensed phase in the presence of a solvent, comprising the steps of:
A) transitioning to a hot phosgenation and
B) expelling residual phosgene and/or hydrogen chloride, characterized in that the reaction is carried out in a batchwise manner in at least two reactors arranged in parallel and in that step A) and/or step B) are carried out asynchronously in at least two of the reactors arranged in parallel.

Both mono-and polyamines, i.e. amines having two or more amino groups per molecule, can be used for the process of the invention. Preference is given to diamines.

Examples of preferred diamines are diaminotoluene (TDA), especially 2,4-diaminoluene and 2,6-diaminotoluene, diaminodimethylbenzene, diaminonaphthalene, especially 1,5-diaminonaphthalene (NDA), diaminobenzene, especially 1,4-diaminobenzene (pPDI), diaminodiphenylmethane (MDA), especially 2,2′-diaminodiphenylmethane, 2,4′-diaminodiphenylmethane and 4,4′-diaminodiphenylmethane, 1,4-diaminobutane, 1,5-diaminopentane (PDA), 1,6-diaminohexane (HDA), 1,11-diaminoundecane, 1-amino-3,5,5-trimethyl-5-aminomethylcyclohexane (IPDA), bis(p-aminocyclohexyl) methane (PACM), 1,5-diamino-2-methylpentane, 2,5-diamino-2,5-dimethylhexane, 1,4-diaminocyclohexane, hexahydrotolylenediamine (H6TDA), especially 2,4-hexahydrotolylenediamine, 2,6-hexahydrotolylenediamine, 1,3-bis(aminomethyl)benzene (m-XDA), 1,4-bis(aminomethyl) benzene (p-XDA), bis(aminomethyl)cyclohexane (H6-XDA), tetramethylxylylenediamine (TMXDA), bis(aminomethyl)norbornane (NBDA), neopentanediamine, 2,4,4-trimethylhexamethylenediamine, 2,2,4-trimethylhexamethylendiamine, and mixtures thereof.

Examples of particularly preferred diamines are 1,5-diaminonaphthalene (NDA), 1,4-diaminobenzene (pPDA), 1,5-diaminopentane (PDA), 1,6-diaminohexane (HDA), 1-amino-3,5,5-trimethyl-5-aminomethylcyclohexane (IPDA), bis (p-aminocyclohexyl) methane (PACM), 1,5-diamino-2-methylpentane, 2,5-diamino-2,5-dimethylhexane, 1,4-diaminocyclohexane, hexahydrotolylenediamine (H6TDA), 1,3-bis(aminomethyl)benzene (m-XDA), 1,4-bis(aminomethyl)benzene (p-XDA), bis (aminomethyl) cyclohexane (H6-XDA), bis(aminomethyl)norbornane (NBDA), and mixtures thereof.

Examples of very particularly preferred diamines are 1,5-diaminonaphthalene (NDA), 1,4-diaminobenzene (pPDA), 1,5-diaminopentane (PDA), bis(p-aminocyclohexyl)methane (PACM), hexahydrotolylenediamine (H6TDA), 1,3-bis(aminomethyl)benzene (m-XDA), bis(aminomethyl)norbornane (NBDA), and mixtures thereof.

Examples of most preferred diamines are 1,5-diaminonaphthalene (NDA), 1,4-diaminobenzene (pPDA), bis(p-aminocyclohexyl)methane (PACM), 1,3-bis(aminomethyl)benzene (m-XDA), bis(aminomethyl)norbornane, and mixtures thereof.

Instead of the amines, it is also possible to phosgenate the salts thereof, especially the hydrochlorides or carbamates, preferably the hydrochlorides, of the amines. These salts are in that case usually produced in situ in a first step at low temperature, as described for example in DE19510259A1, it being optionally possible to dispense with the introduction of the inert gas. It is preferable to phosgenate the amines directly (base phosgenation), specifically in a two-stage reaction at different reaction temperatures (cold-hot phosgenation).

The reaction is carried out in the presence of a solvent. All solvents known to those skilled in the art that are inert or at least largely inert under the prevailing reaction conditions are suitable. Preferred solvents are selected from the group consisting of aromatic hydrocarbons, halogenated aromatic hydrocarbons, especially chlorinated aromatic hydrocarbons, esters, ethers, halogenated hydrocarbons, and mixtures thereof. Aromatic hydrocarbons used with particular preference according to the invention are selected from the group consisting of toluene, bromobenzene, chlorobenzene, dichlorobenzene, especially o-dichlorobenzene, and mixtures thereof. According to the invention, particular preference is given to using chlorobenzene, o-dichlorobenzene or mixtures of these two solvents.

The reaction is carried out in a batchwise manner. This is in the present invention to be understood as meaning that the liquid reaction product is not continuously withdrawn from the reactors, but that the liquid reaction product is withdrawn from a reactor, and transferred to a holding vessel for the crude product or sent directly for further processing, essentially only once conversion into the isocyanate in said reactor is complete. Those skilled in the art are familiar with various methods for determining the end point of the reaction. It is usually what is known as the “clear point” that is used, this being the point at which a clear solution has formed from the suspension initially present in the hot phosgenation. As an alternative, it is also possible to use the end of evolution of HCl gas or the presence of a constant NCO content in the reaction mixture. Even during the reaction, it is however possible to withdraw a portion of the liquid reaction mixture from the reactor and return it back thereto, for example in order to bring about better mixing or to control the temperature of the reaction mixture without contravening the condition of batchwise phosgenation in the context of the present invention. Gaseous flows comprising essentially hydrogen chloride, phosgene, inert gas and solvent vapors may however be withdrawn from the reactors and reprocessed at any time, including continuously. Preferably, the reaction is carried out in what is known as a semi-batchwise process, also referred to as fed-batch process or feed process, that is to say the initially partially filled reactor is fed during the reaction with amine and/or phosgene and optionally inert gas.

Stirred-tank reactors are particularly suitable as reactors, but other types of reactors, such as loop reactors, can also be used in principle. According to the invention, the reaction of amine with phosgene is carried out in at least 2 reactors arranged in parallel, preferably in 2 to 10 reactors arranged in parallel, more preferably in 2 to 6 reactors arranged in parallel, and most preferably in 2 or 4 reactors arranged in parallel. The reaction is preferably carried out at an absolute pressure of from 0.100 MPa to 2.000 MPa, preferably 0.105 MPa to 1.000 MPa, and more preferably from 0.110 MPa to 0.600 MPa. Accordingly, the reactors are preferably equipped with a pressure-retaining device, for example a control valve, via which process offgas can escape from the reactor.

In a base phosgenation, that is to say the reaction of the amines with phosgene, the reaction is preferably carried out in two stages in the inert solvent. Such reactions are described for example in W. Siefken, Liebigs Annalen der Chemie, 562 (1949) p. 96. In the first stage, the cold phosgenation, the temperature of the reaction mixture is preferably kept within a range ≥0° C. and <100° C. A suspension comprising the carbamoyl chloride, amine hydrochloride, and small amounts of free isocyanate forms. The preferred procedure here is for a solution of phosgene in an inert solvent to be initially charged and a solution or suspension of the amine in the same solvent and optionally further phosgene then added. This keeps the concentration of free amine low and thus suppresses the undesired formation of ureas.

In the second stage, the hot phosgenation, the temperature is increased and is then preferably within a range of from 120° C. to 200° C. This heating of the reaction mixture to a temperature above 100° C. carried out at the start of the hot phosgenation is to be understood in the present case as meaning step (B), “transitioning to the hot phosgenation”. The temperature is then preferably kept within this range, preferably during at least the time in which further phosgene is supplied until the reaction to the isocyanate has ended, i.e. until the evolution of HCl ceases and/or the reaction mixture becomes clear. Phosgene is advantageously used in excess for the reaction. If required, the reaction may be carried out with introduction of an inert gas both in the cold phosgenation and in the hot phosgenation.

If the amines to be converted are high-melting amines that are sparingly soluble in the solvent, it is also possible to use a suspension of the amine for the phosgenation. This is preferably produced by dispersion with a dynamic mixing unit, as described in EP2897933B1, paragraph [0020] to paragraph [0024].

In the amine hydrochloride or carbamate phosgenation, the amine is preferably initially reacted with hydrogen chloride gas or carbon dioxide in an inert liquid medium to produce the corresponding salt. The reaction temperature during this salt formation is preferably within a range of from 0 to 80° C. An initial reaction with phosgene can already take place subsequently or at the same time. This is followed by a further phosgenation step that is essentially similar to the hot phosgenation from the base phosgenation described above and is therefore likewise referred to hereinafter as hot phosgenation. Here too, the temperature is thus preferably kept within the range from 120 to 200° C. and while preferably phosgene and optionally an inert gas are being introduced into the reaction mixture. The introduction is continued preferably until the reaction to the isocyanate has ended. Here too, phosgene is preferably employed in excess in order to speed up the reaction.

The phosgenation is usually carried out with a stoichiometric excess of phosgene, that is to say more than one mole phosgene per mole of amino groups is used. The molar ratio of total phosgene used to amino groups is preferably 1.02:1 to 20.0:1, more preferably 1.1:1 to 10.0:1, and most preferably 1.2:1 to 5.0:1. If necessary, further phosgene or phosgene solution can be supplied to the reaction mixture during the reaction in order to maintain a sufficient excess of phosgene or to compensate for loss of phosgene.

Both in the base phosgenation and in the phosgenation of the amine hydrochloride or carbamate, the residual phosgene and hydrogen chloride gas is removed at the end of the reaction, preferably by purging with an inert gas, preferably with nitrogen. If necessary, the reaction mixture may be filtered to remove any solids present, such as unreacted amine hydrochlorides.

In the course of batchwise phosgenation, at least two maxima normally occur in the mass flow of process offgas for each batch. An increase in the mass flow of process offgas from a reactor occurs for example during the transition to the hot phosgenation. The heating of the reaction mixture lowers the solubility of gases in the mixture, resulting in outgassing of dissolved gases and a consequent increase in the offgas stream from the reactor, which, depending on the selected process, comprises phosgene, hydrogen chloride and/or carbon dioxide in particular. A second maximum occurs at the end of the reaction when residual phosgene and hydrogen chloride are expelled from the reaction mixture and—if the reaction has been carried out under pressure—the reactor is depressurized. To permit a more economical design of the apparatuses and operating conditions for process offgas reprocessing, according to the invention, at least two of the reactors are operated asynchronously to one another, i.e. at least one reactor is operated asynchronously to at least one of the other reactors.

This means that step A) transitioning to the hot phosgenation, i.e. the heating of the reaction mixture to above 100° C., and/or step B) expelling residual phosgene and/or hydrogen chloride, which also optionally includes the depressurization of the reactor to a lower pressure, is carried out in this at least one reactor with a time delay or under different conditions, especially at a different rate, than in at least one of the other reactors. It is for example possible during the transition to the hot phosgenation to execute the same temperature ramp with a time delay in the at least two asynchronously operated reactors or else, in at least one reactor, to execute a different temperature ramp, especially a flatter one, with or without a time delay so that the associated maximum in the offgas mass flow is lower and/or does not coincide with the maximum in the offgas mass flows of the other reactors. Particularly preferably, the asynchronously operated reactors execute the same temperature ramp with a time delay, so that the temperature increase in the transition to the hot phosgenation does not occur simultaneously. Preferably, the time delay is at least 5 minutes, more preferably at least 10 minutes, and most preferably at least 20 minutes. Likewise preferably, the time delay is not more than 24 h, preferably not more than 8 hours, more preferably not more than 2 h, and most preferably not more than 1 h. Where the periods in which the temperature increases in the at least two of the reactors arranged in parallel in which the transition to the hot phosgenation takes place asynchronously overlap, it is in each case the time at which a temperature of 100° C. in the reaction mixture is exceeded that is key and the time delays mentioned relate to this time.

The same also holds for step B) expelling residual phosgene and/or hydrogen chloride. In this case, it is for example possible for the depressurization and/or optional introduction of inert gas to be carried out asynchronously in at least two of the reactors arranged in parallel, i.e. with a time delay and/or at different rates, in order in turn to ensure that the associated maximum in the offgas mass flow is lower and/or does not coincide with the maximum in the offgas mass flows in the other reactors. Particularly preferably, the depressurization and/or optional introduction of inert gas is carried out with the same specifications regarding the pressure profiles and inert gas mass flows over time as for the other reactors, but with a time delay. Preferably, the expulsion of phosgene and/or hydrogen chloride is carried out with a time delay of at least 5 minutes, more preferably at least 10 minutes, and most preferably at least 20 minutes. Likewise preferably, the time delay is not more than 24 h, preferably not more than 8 hours, more preferably not more than 2 h, and most preferably not more than 1 h.

In a further preferred embodiment of the invention, the at least two of the reactors arranged in parallel in which step A) and/or step B) are carried out asynchronously are operated according to the same operating instructions, but the individual process steps are run through with a time delay of at least 5 minutes, preferably at least 10 minutes, and more preferably at least 20 minutes.

In a further preferred embodiment, at least three, preferably at least four, and more preferably all, of the reactors arranged in parallel are operated asynchronously to one another. Preferably, all reactors are operated according to the same operating instructions, but the individual process steps are run through with a time delay relative to one another. Preferably, the time delay is at least 5 minutes, more preferably at least 10 minutes, and most preferably at least 20 minutes. Likewise preferably, the time delay is not more than 24 h, preferably not more than 8 hours, more preferably not more than 2 h, and most preferably not more than 1 h.

In addition, it is also advantageous to stagger the reactors such that transitioning to the hot phosgenation, that is to say the increase in temperature to over 100° C., is not underway in any of the reactors while expulsion of phosgene and hydrogen chloride or depressurization is taking place in another reactor.

The process offgas normally comprises phosgene, hydrogen chloride, solvent vapors and also any inert gas and traces of other volatile components of the reaction mixture, such as amines or isocyanates. In a further preferred embodiment, process offgas streams are withdrawn from the reactors and at least partially combined into at least one process offgas stream from which phosgene is recovered. The recovered phosgene is particularly preferably reused for the reaction of amine to isocyanate. The process offgas streams of the various reactors are preferably merged in one collecting line and then together supplied to a reprocessing in which phosgene is recovered from the collected process offgas.

In a preferred embodiment of the invention, the recovery of phosgene from the at least one process offgas stream comprises at least one absorption step in which the phosgene is scrubbed from the process offgas stream by means of at least one absorbent in one or more absorption devices, affording an offgas stream and a phosgene solution. In this case, the process offgas is contacted with an absorbent so as to scrub phosgene from the process offgas and to generate a hydrogen chloride-containing offgas stream that is as phosgene-depleted as possible.

Such an absorption step can generally be carried out in one or more suitable absorption devices. Suitable absorption devices are for example gas scrubbers, in which the process offgas stream is contacted with a liquid stream, also termed absorbent or scrubbing medium, examples of said scrubbers being immersion scrubbers, spray scrubbers, packed columns with a random packing, packed columns with a structured packing, tray columns or falling-film absorbers. For efficient absorption of phosgene from the process offgas stream it is also possible to combine different types of gas scrubber. Preference here is given to combining two or more, more preferably two, gas scrubbers in an absorption device. Preferably, the absorption device comprises a gas scrubber selected from the group consisting of packed columns with a random packing, packed columns with a structured packing, and tray columns, more preferably consisting of packed columns with a random packing and packed columns with a structured packing.

A particularly suitable absorption device comprises a falling-film absorber that is suitable for carrying out an isothermal absorption and a tray column, packed column with a random packing or packed column with a structured packing, preferably a packed column with a random packing or packed column with a structured packing, more preferably a packed column with a structured packing, that is suitable for carrying out an adiabatic absorption. A falling-film absorber is preferably a vertically arranged shell-and-tube heat exchanger in which the process offgas and the absorbent are conducted either on the tube side or on the shell side of the heat exchanger, preferably on the tube side of the heat exchanger, while on the other side in each case a cooling medium is conducted in cocurrent or countercurrent, preferably in countercurrent, to the process offgas stream. The process offgas and the absorbent can equally be conducted in cocurrent or countercurrent to one another, preferably in countercurrent to one another. The tubes can optionally be furnished with a random or structured packing or other internals to improve mass transfer and/or heat exchange. The tray column, packed column with a random packing or packed column with a structured packing is preferably set up such that the process offgas from the isothermal gas scrubber is in countercurrent contact with the absorbent. The absorbent is preferably introduced at the upper end of the column via a liquid distributor. The packed column is preferably one having a structured packing. Particularly preferably, the falling-film absorber and the tray column, packed column with a random packing or packed column with a structured packing are combined in a column-form apparatus that includes at the lower end a supply line for the process offgas. The falling-film absorber is arranged thereabove, and above that the tray column, packed column with a random packing or packed column with a structured packing. In order that the absorbent is distributed evenly on the tubes/on the column, liquid distributors are preferably present above the falling-film absorber and at the head of the column-form apparatus. Optionally present at the head of the absorption device is an internal or external condenser for further cooling of the offgas stream that is now depleted of phosgene.

The offgas stream, which is as phosgene-depleted as possible, usually consists essentially, i.e. to an extent of at least 80% by volume, preferably to an extent of at least 95% by volume, and more preferably to an extent of at least 98% by volume, of hydrogen chloride. It also contains residual solvent, inert gases if used, and traces of phosgene. The phosgene content of this offgas stream is preferably not more than 1.0% by volume, more preferably not more than 0.5% by volume, and most preferably not more than 0.2% by volume. The natural and generally desirable lower limit for the phosgene content in this offgas stream is 0.00% by volume. However, not only can adherence to this limit lead to increased consumption of absorbent, but it may also be advantageous for the design of a closed-loop control unit to permit a low phosgene content in the offgas, for example in order to use this as a closed-loop control variable. Therefore, the phosgene content in the offgas stream is preferably at least 0.01% by volume, more preferably at least 0.02% by volume, and most preferably at least 0.05% by volume.

In order to achieve maximum efficiency of absorption, it is advantageous to cool the process offgas initially to a temperature in the range from 5° C. to −25° C., preferably in the range from 0° C. to −20° C., and more preferably in the range from −5° C. to −15° C. The cooling can be effected simply by means of one or more heat exchangers or by quenching, i.e. by contacting the process offgas with an already cooled liquid in a quench device. This can be done, for example, in a gas scrubber, preferably a spray scrubber, or in an irrigated heat exchanger. A suitable cooled liquid is for example the absorbent. Preferably, the absorbent here, after contact with the process gas, is deposited as a liquid and this liquid is recirculated into the quench device, where the liquid loop or the quench device itself contains a cooler to cool the liquid to the desired temperature. During cooling, partial condensation/absorption of phosgene occurs.

The residual gas phase is then contacted with the absorbent, preferably in countercurrent. At the same time, the rising gas phase is preferably contacted in countercurrent with the descending liquid absorbent. Suitable devices for this purpose are known to those skilled in the art. Preferably, the absorption comprises a combination of two absorption steps, the gas preferably first passing through an isothermal absorption and then an adiabatic absorption. The volume flow of the absorbent is preferably variable and can in each case be adapted to the prevailing process conditions and the resulting requirements. The absorbent used is preferably the same solvent that was also present during the reaction of the amine or salt thereof with phosgene. Particular preference is given to using chlorobenzene, o-dichlorobenzene or a mixture of the two as absorbent. In the preferred embodiment with two absorption steps, fresh or reprocessed absorbent is preferably introduced in the second absorption step, i.e. the absorption step that the gas passes through later, whereas in the first absorption step preference is given to using already-laden absorbent from the second absorption step, optionally together with fresh absorbent and/or a recirculated, likewise already-laden absorbent from the first absorption step and/or the preceding cooling process. Preferably, the gas stream after contact with the absorbent passes through a condenser in order to condense absorbed absorbent and to separate it as completely as possible from the gas stream.

The laden absorbent can, as previously described, be employed as a quench medium in order to initially cool the process offgas stream. A portion of the absorbent is withdrawn either continuously or discontinuously and, optionally after addition of further phosgene and/or solvent, is reused for isocyanate production, preferably in one of the reactors for phosgenation of the amine or salt thereof. The withdrawn, laden absorbent is preferably a solution containing 20% by weight to 70% by weight, more preferably 30% by weight to 68% by weight, and most preferably 42% by weight to 66% by weight, of phosgene in chlorobenzene. The content of the solution here depends in particular on the phosgene content of the process offgas, on the amount of fresh absorbent for absorption, and on the amount of absorbent withdrawn.

In a particularly preferred embodiment of the process of the invention, the mass flow of absorbent into the absorption device is preferably automatically controlled, preferably by closed-loop control. For this purpose, a target value for the mass flow of absorbent into the absorption device is determined and translated into a control variable for an actuator that influences the mass flow. The determination of the target value for the mass flow takes account of at least one of the following measured variables a) to d) or its change over time:
a) the phosgene content of the offgas stream obtained downstream of the at least one absorption step
b) the mass flow of process offgas into the absorption device
c) the concentration of the phosgene solution obtained in the absorption step
d) the content of hydrogen chloride in the process offgas flow into the absorption device.

Such a procedure is particularly suitable in a batchwise reaction regime, in which fluctuations in the amount and composition of the process offgas inevitably occur. A recommendation from the prior art is for the amount of absorbent to be guided by the mass flow of phosgene to the absorbent device. For example, DE102008009761 A1, paragraph [0048], and US2007/0249859 A1, paragraph [0048], disclose weight ratios of solvent (absorbent in the present case) and phosgene at the inlet to the absorption of 0.1:1 to 3:1:1, preferably 1:1 to 10. How to deal with fluctuations in the amount of phosgene and in the total mass flow of process offgas at the inlet to the absorption is not discussed further, since these documents primarily concern continuous processes in which such fluctuations do not occur or occur only to a much lesser degree. However, operational practice has shown that the application of this criterion alone, especially in batchwise operation of a single reactor or of a plurality of synchronously operated reactors, is not enough for the process to be operated with satisfactory effectiveness and efficiency. In batchwise operation, penetration of phosgene into the hydrogen chloride-containing offgas stream downstream of the absorption occurs time and again. Basing a design of the absorption device on peak loading is also problematic, because in periods of lower loading a high amount of absorbent would then still have to be expended in order to maintain sufficient liquid loading for good mass transfer. This in turn leads to significant fluctuations in the concentration of phosgene in the absorbent and as a result makes it difficult to reuse the obtained phosgene solution in the phosgenation reaction or makes the reprocessing of the absorbent more laborious.

In order to achieve maximum constancy of quality of the phosgene-depleted offgas stream downstream of the absorption, it is preferable to take account of the phosgene content of the offgas stream obtained downstream of the at least one absorption step in determining the target value for the mass flow of absorbent. This can be done for example by determining the content of phosgene in said offgas stream and comparing it against a target value. If the content is above the target value, the target value for the mass flow of absorbent to the absorption device is increased. If the content is below the target value, the mass flow of absorbent to the absorption device is lowered. Methods for determining the phosgene content in the offgas stream are known to those skilled in the art. The determination can be carried out for example by IR filter photometry, for example using a multicomponent process photometer.

The invention therefore further provides a computer-implemented process for adjusting a mass flow of absorbent in an absorption device for reprocessing phosgene-containing process offgas streams, in which a target value for the mass flow of absorbent is determined and translated into a control variable for an actuator that influences the mass flow, where at least one of the following measured variables or its change over time is taken into account in determining the target value for the mass flow:
the phosgene content of the offgas stream obtained downstream of the at least one absorption step
the mass flow of process offgas into the absorption device
the concentration of the phosgene solution obtained in the absorption step
the content of hydrogen chloride in the process offgas flow into the absorption device.

In a preferred embodiment of the computer-implemented process, the phosgene content of the offgas stream obtained downstream of the at least one absorption step is taken into account in determining the target value for the mass flow of absorbent.

### EXAMPLES

### Comparative Example 1

A solution of 60 kg of phosgene in 150 kg of chlorobenzene was prepared at 0° C. in each of two parallel-installed 0.5 m³ stirred-tank reactors with temperature control unit and fitted with a reflux condenser. After passing through the reflux condensers, the offgas from the reactors was brought together in a collecting line. To the phosgene solution in each of the two reactors was simultaneously added, with stirring, a suspension of 25 kg of 1,5-diaminonaphthalene in 75 kg of chlorobenzene. The suspension was stirred for approx. 120 minutes without further cooling prior to the transition from the cold phosgenation phase to the hot phosgenation phase. For this, the reaction mixture in the two reactors was simultaneously heated and kept under reflux for about 6 h. The pressure during the reaction was approx. 2.9 bar (a). After the reaction mixture had become clear, which signals the end of the phosgenation reaction, the reactors were first depressurized and the liquid crude product was run off and the isocyanate isolated in a distillation sequence.

The offgas from the collecting line was at the same time supplied to a two-stage absorption column in which it was washed with chlorobenzene in countercurrent. The process offgas here passed through first an isothermal absorption stage at approx. −15° C. and then an adiabatic absorption stage, before exiting the process as offgas. Fresh chlorobenzene was added at −5° C. at the upper end of the adiabatic absorption stage. The mass flow of fresh chlorobenzene to the absorption column was approx. 36 kg/h. After passing through the adiabatic absorption stage, the solution of phosgene in chlorobenzene obtained therein was forwarded as a wash solution to the isothermal absorption stage, with the result that a solution of phosgene in MCB was ultimately withdrawn at the bottom of the two-stage absorption column, and this, after adjusting the phosgene concentration, served as a starting material for further phosgenations. The concentration of the withdrawn phosgene solution decreased in the course of the reaction, which meant that an increasing amount of fresh phosgene needed to be added to adjust the phosgene concentration for further use. At the head of the absorption column, a hydrogen chloride stream largely freed of phosgene was obtained. However, during the reaction there was short-lived penetration of a small amount of phosgene into the offgas of the absorption device, making the further treatment of the process offgas more laborious.

During the production run, a maximum process offgas mass flow of approx. 70 kg/h was observed in the collecting line. This occurred during the transition from the cold phosgenation phase to the hot phosgenation phase, and the process offgas consisted of approx. 90% phosgene and approx. 10% hydrogen chloride. After passing through this maximum, the overall mass flow of process offgas decreased and the composition shifted toward higher hydrogen chloride contents. At the end of the reaction, the process offgas stream ceased. There was one more short-lived increase in the mass flow of process offgas in the collecting line solely when the pressure in the reactors was released, but this was much less pronounced than the first maximum.

### Comparative Example 2

The synthesis from comparative example 1 was repeated under the same reaction conditions. Unlike in comparative example 1, the absorption device was in this case not charged with a constant mass flow of chlorobenzene. Instead, this was varied as a function of the phosgene mass flow in the process offgas, such that the mass flow of fresh hydrogen chloride into the absorption column always corresponded to 1.3 times the mass flow of phosgene into the absorption column, provided that the irrigation density of the absorption column was not below the minimum value. The maximum mass flow of chlorobenzene during the transition from the cold phosgenation to the hot phosgenation, i.e. at the point of greatest phosgene mass flow in the process offgas, was 82 kg/h. During the hot phosgenation, about 1 h after the process offgas mass flow had reached its maximum, penetration of phosgene into the offgas of the absorption device occurred, which intensified in the following 1.5 h. Over the total run time of batchwise production, the consumption of chlorobenzene was higher than in example 1, with the result that the concentration established in the phosgene solution obtained at the bottom of the column was on average lower. Moreover, the penetration of phosgene into the offgas of the absorption column was found to be more pronounced than in comparative example 1.

### Example 1 (According to the Invention)

The synthesis from example 1 was repeated with the difference that the second reactor was operated with a time delay of 3 hours, i.e. the addition of the amine suspension and also the heating for the transition from cold phosgenation to the hot phosgenation in the second reactor were not carried out synchronously with the first reactor, but with said time delay of 3 hours. In addition, the mass flow of fresh chlorobenzene to the absorption column was set at a constant 30 kg/h.

During the transition of the first reactor from the cold to the hot phosgenation, a first maximum in the process offgas mass flow in the collecting line was observed. The mass flow at this point was approx. 35 kg/h and the process offgas again consisted of approx. 90% phosgene and approx. 10% hydrogen chloride. In the further course of production, the process offgas mass flow passed through a second, higher maximum of approx. 46 kg/h. This occurred during the transition in the second reactor from the cold phosgenation phase to the hot phosgenation phase, and the process offgas consisted of approx. 79% phosgene and approx. 21% hydrogen chloride. After passing through this maximum, the overall mass flow of process offgas decreased and the composition shifted toward higher hydrogen chloride contents. Since the depressurization of the two reactors after the reaction had also been carried out with a corresponding time delay, the process offgas mass flow in the collecting line likewise showed not one, but two further brief and less pronounced peaks.

Despite the reduced use of chlorobenzene in the absorption column compared to comparative example 1 and comparative example 2, penetration of phosgene into the offgas of the absorption column was not observed at any point in this experiment. The content of phosgene in the phosgene solution withdrawn at the bottom of the absorption column fluctuated, but was on average higher than in the previously described examples 1 and 2. Both the maximum gas loading and the liquid loading of the absorption column were reduced compared to examples 1 and 2, thereby making possible the use of an absorption column having a smaller diameter and consequently lower capital costs.

### Example 2 (According to the Invention)

The synthesis from example 1 was repeated with time-delayed operation of the reactors. However, unlike in example 1, the absorption device was in this case not charged with a constant mass flow of chlorobenzene. Instead, the phosgene content in the offgas from the absorption device was tracked by online measurement. The target value for the phosgene content was set at 0.05% by volume and the deviation of the measured value from this target value acted, via a closed-loop control circuit, ultimately on a control valve in the fresh chlorobenzene supply line, so as to adjust the mass flow of chlorobenzene to the absorption column accordingly and to keep the deviation as small as possible.

Over the total run time of batchwise production, the consumption of chlorobenzene in this example was again lower than in example 1, with the result that a higher concentration was established on average in the phosgene solution obtained at the bottom of the column, while the offgas from the absorption column was only minimally contaminated with phosgene over the entire run time. The content of phosgene here was low and constant, and so further reprocessing to destroy residual phosgene is simple.

1. A process for producing isocyanates by reacting at least one amine or salt thereof with a stoichiometric excess of phosgene in the condensed phase in the presence of a solvent, comprising the steps of:
A) transitioning to a hot phosgenation and
B) expelling residual phosgene or hydrogen chloride,
wherein the reaction is carried out in a batchwise manner in at least two reactors arranged in parallel and in that step A) or step B) are carried out asynchronously in at least two of the reactors arranged in parallel.

2. The process of claim 1, wherein the at least one amine is at least one diamine, preferably 1,5-diaminonaphthalene (NDA), 1,4-diaminobenzene (pPDA), 1,5-diaminopentane (PDA), bis(p-aminocyclohexyl)methane (PACM), hexahydrotolylenediamine (H6TDA), 1,3-bis(aminomethyl)benzene (m-XDA) or bis (aminomethyl) norbornane (NBDA), or mixtures thereof.

3. The process of claim 1, wherein the reaction is carried out in a semi-batchwise process.

4. The process of claim 1, wherein the reaction is carried out in 2 to 10 reactors arranged in parallel.

5. The process of claim 1, wherein at least three, preferably at least four, and more preferably all, of the reactors arranged in parallel are operated asynchronously to one another.

6. The process of claim 1, wherein step A) transitioning to a hot phosgenation is carried out asynchronously in at least two of the reactors.

7. The process of claim 1, wherein process offgas streams are withdrawn from the reactors and at least partially combined into at least one process offgas stream from which phosgene is recovered.

8. The process of claim 1, wherein process offgas streams are withdrawn from the reactors and at least partially combined into at least one process offgas stream from which phosgene is recovered, wherein the recovery of phosgene from the at least one process offgas stream comprises at least one absorption step in which the phosgene is scrubbed from the process offgas stream by means of at least one absorbent in one or more absorption devices, producing an offgas stream and a phosgene solution.

9. The process of claim 8, wherein a solvent employed as absorbent in the at least one absorption step is preferably the solvent that is also present in the reaction of the at least one amine or salt thereof.

10. The process of claim 8, wherein the absorption device comprises a gas scrubber selected from the group consisting of packed columns with a random packing, packed columns with a structured packing, and tray columns.

11. The process of claim 8, wherein the absorbent is added in a mass flow, the target value for which is determined taking account of at least one of the following measured variables a) to d) or its change over time:
a) the phosgene content of the offgas stream obtained downstream of the at least one absorption step,
b) the mass flow of process offgas into the absorption device,
c) the content of hydrogen chloride in the process offgas flow into the absorption device, or
d) the concentration of the phosgene solution obtained in the absorption step.

12. The process of claim 8, wherein the phosgene solution obtained in the absorption device is reused for isocyanate production.

13. The process of claim 11, wherein the phosgene content of the offgas stream obtained downstream of the at least one absorption step is taken into account in determining the target value for the mass flow of absorbent.

14. A computer-implemented process for adjusting a mass flow of absorbent in an absorption device for reprocessing phosgene-containing process offgas streams, in which a target value for the mass flow of absorbent is determined and translated into a control variable for an actuator that influences the mass flow, wherein at least one of the following measured variables or its change over time is taken into account in determining the target value for the mass flow:
the phosgene content of the offgas stream obtained downstream of the at least one absorption step,
the mass flow of process offgas into the absorption device,
the concentration of the phosgene solution obtained in the absorption step, or
the content of hydrogen chloride in the process offgas flow into the absorption device.

15. The process of claim 14, wherein the phosgene content of the offgas stream obtained downstream of the at least one absorption step is taken into account in determining the target value for the mass flow of absorbent.

16. The process of claim 5, wherein at least four of the reactors arranged in parallel are operated asynchronously to one another.

17. The process of claim 5, wherein all of the reactors arranged in parallel are operated asynchronously to one another.

18. The process of claim 4, wherein the reaction is carried out in 2 to 6 reactors arranged in parallel.

19. The process of claim 4, wherein the reaction is carried out in 2 to 4 reactors arranged in parallel.

20. The process of claim 2, wherein the at least one amine selected from the group consisting of 1,5-diaminonaphthalene (NDA), 1,4-diaminobenzene (pPDA), 1,5-diaminopentane (PDA), bis(p-aminocyclohexyl)methane (PACM), hexahydrotolylenediamine (H6TDA), 1,3-bis(aminomethyl)benzene (m-XDA) or bis(aminomethyl)norbornane (NBDA), and mixtures thereof.
